(19) United States
(12) Patent Application Publication
(10) Pub. No.: US 2026/0242404 A1
(43) Pub. Date: Aug. 20, 2026

(54) INTERGLYCAN ESTERS OF SUCROSE DICARBOXYLATE AND FRUCTOFURANURONATE, PROCESS FOR THE MANUFACTURE THEREOF, AND USE THEREOF AS A HYPOGLYCEMIC SLOW SUGAR

(71) Applicant: Abdessattar BOUGOUFFA, SFAX (TN)

(72) Inventor: Abdessattar BOUGOUFFA, SFAX (TN)

(21) Appl. No.: 18/880,003

(22) PCT Filed: Jul. 14, 2022
(86) PCT No.: PCT/TN2022/050003

§ 371 Date: Dec. 30, 2024

(30) Foreign Application Priority Data
Jun. 28, 2022 (TN) TN2022/0175

(51) Int. Cl. C07H 13/10; C07H 1/00

(52) U.S. Cl. CPC C07H 13/10; C07H 1/00

(57) ABSTRACT

The present invention relates to a novel granulated or powdered white natural slow sugar and to a process for manufacturing same based on esterification. The product obtained, i.e. the novel white natural slow sugar, can be consumed by anyone regardless of their state of health because it has a low glycemic index between 15 and 36.5. Obtaining this novel white natural slow sugar by esterification is characterized by slow limited athermal reactions. The novel white natural slow sugar obtained according to the present invention can be used in all food preparations for which traditional white commercial sugar is normally used, such as cakes, pastries, confections, sorbets and ice cream, juices, all types of beverages, chocolate-based preparations, caramel-based cocoa preparations. The novel white natural slow sugar can prevent dental cavities, obesity, retinopathy and other pathologies.

This invention relates to a new natural white granulated or powdered slow sugar, and a process for its manufacture. It is obtained through an industrial manufacturing process based on esterification and in accordance with the FIGURE below including the new molecule of the new slow sugar obtained from commercial white natural sugar (sucrose).

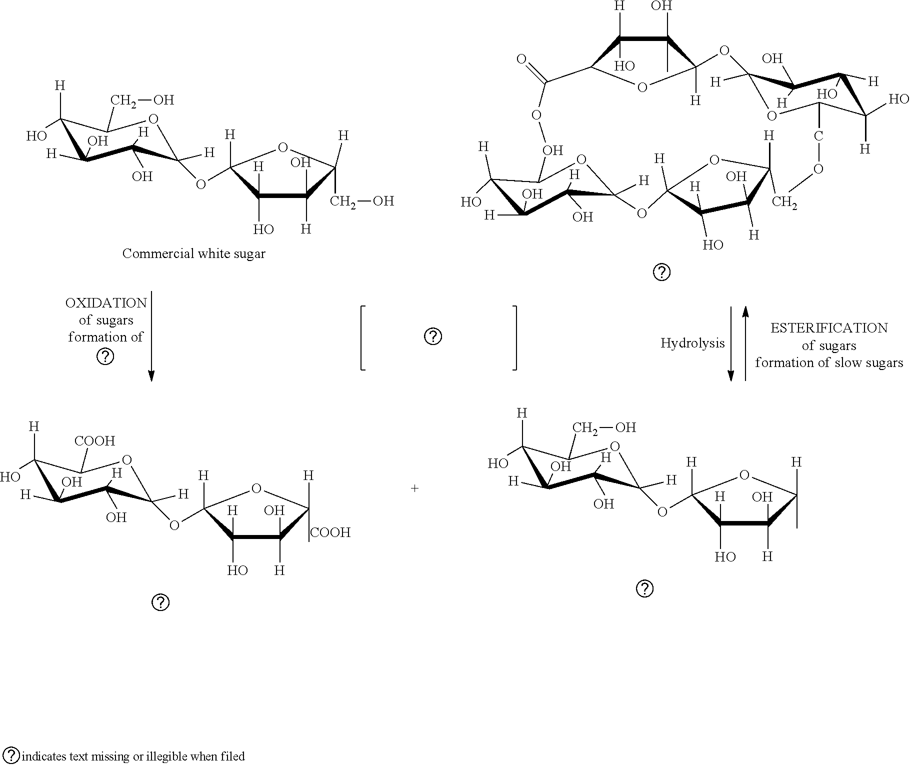

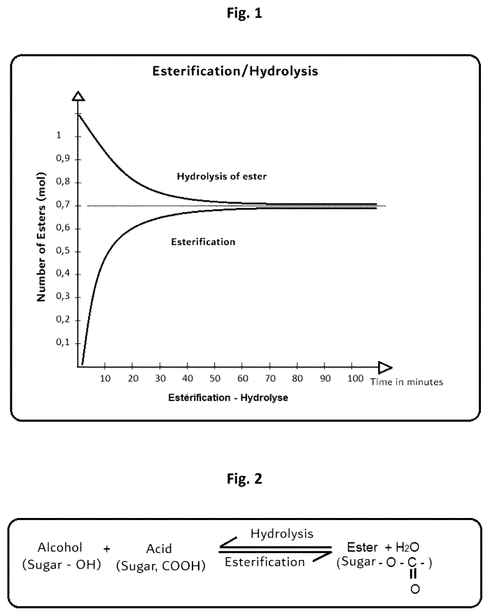

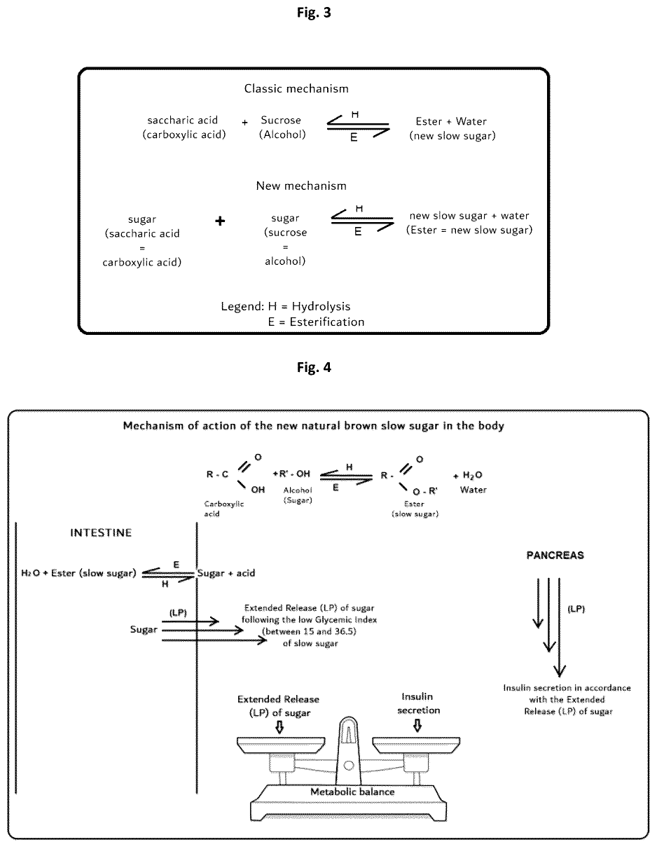

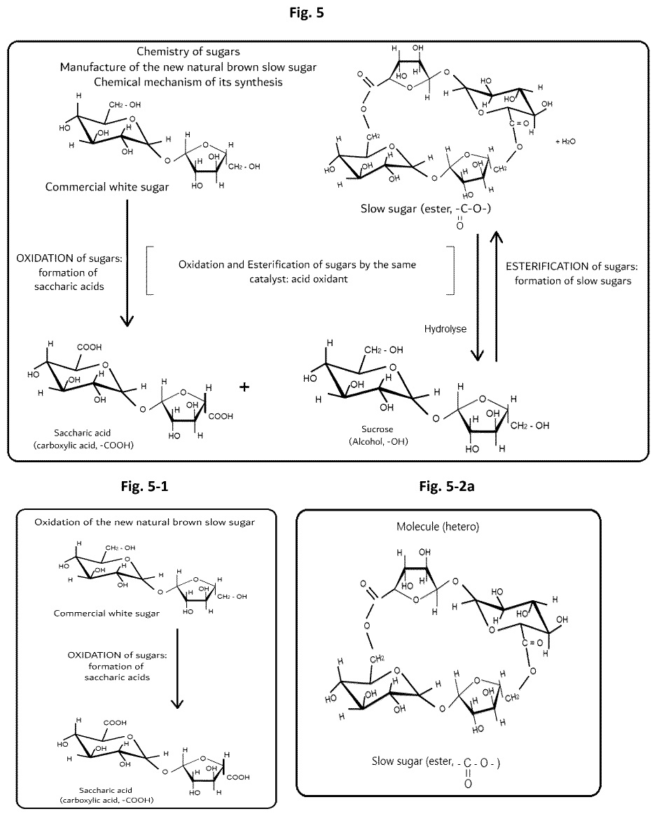

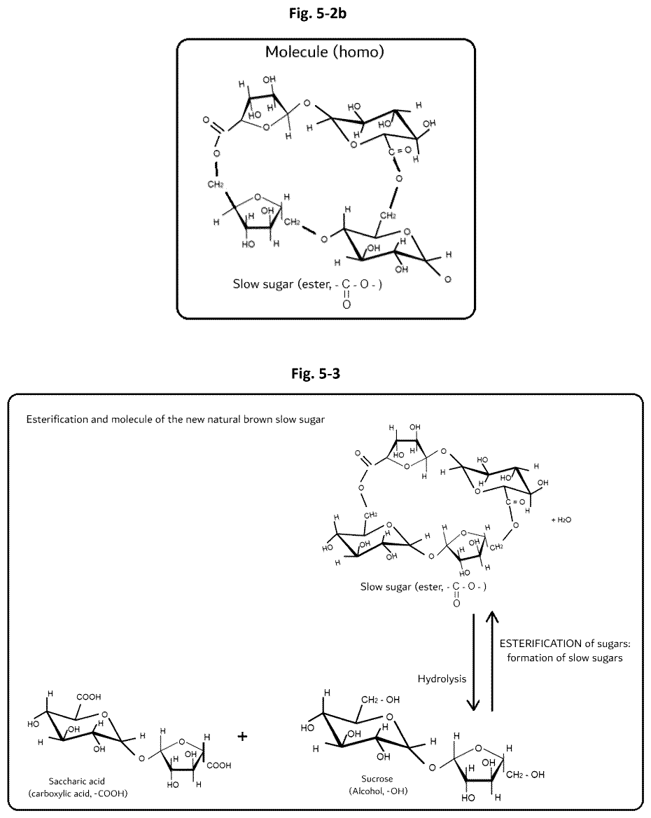

Esterification is a chemical reaction between carboxylic acid and an alcohol that leads to the formation of esters and water (FIG. 1), (FIG. 2), (FIG. 3) and (FIG. 5).

Numerous experimental studies have shown that an equimolar mixture of a carboxylic acid and an alcohol leads to a slow and incomplete transformation.

The esterification reaction of the present invention is described as slow, limited and athermal.

The characteristic of the esterification/hydrolysis equilibrium is the dynamic equilibrium in which the two reverse reactions occur simultaneously and the reactants and products coexist in constant proportions. In fact, the two reactions evolve towards the same equilibrium state.

This invention is based on obtaining a new natural white slow sugar by esterification characterized by slow, limited, and athermal reactions.
Slow since it is characterized by a release of new slow sugar at the level of the intestine and this in concordance with the slow secretion of insulin in the case of type 2 diabetes.
Limited by their reverse reactions since esterification is limited by the hydrolysis of the ester formed.
Athermal because the temperature does not influence the equilibrium.

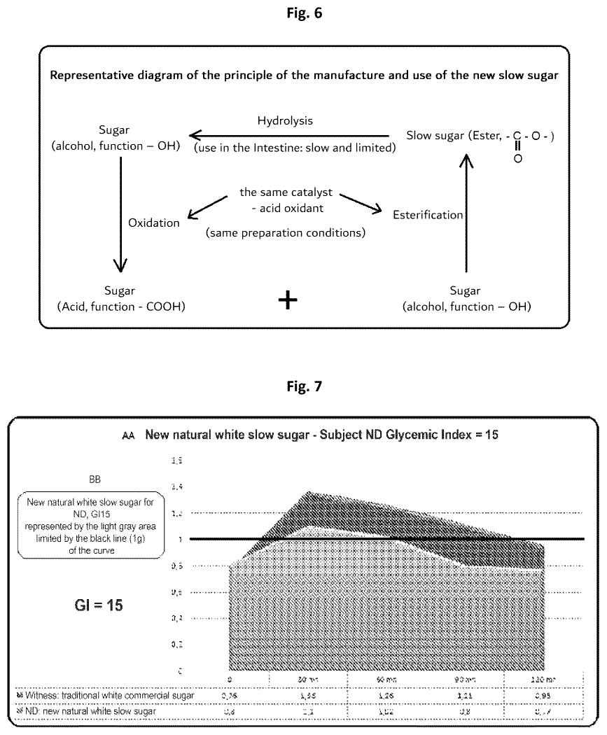

The new natural white granulated or powdered slow sugar of this invention is obtained from commercial white natural sugar, by esterification on an industrial scale and this, by the addition of an acid oxidant, main ingredient and following a manufacturing process (FIG. 6), have made it possible to obtain a new natural white slow sugar acting on consumption by hydrolysis.

Esterification on one side and hydrolysis on the other have achieved a chemical balance in the body, thus making it possible to control consumption by the limited nature of the absorption of this sugar which is controlled by the chemical balance resulting from the esterification/hydrolysis ratio (FIG. 4).

So, if we consume more sugar than the body needs, the excess sugar is not stored but rather eliminated because esterification and hydrolysis perform the permanent control function.

This invention relates to a new natural white slow sugar, the principle of its manufacture of which is based on the chemical balance of esterification of an alcohol and its hydrolysis.

The chemical equilibrium between esterification and hydrolysis is for esterification (FIG. 1), (FIG. 2), (FIG. 3) and (FIG. 4):

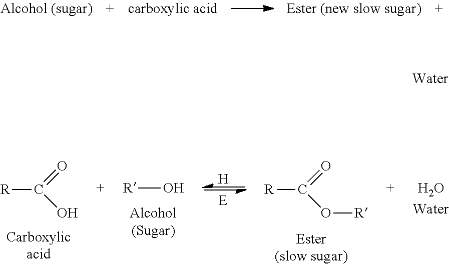

The reaction is a dynamic equilibrium between two reactions (esterification/hydrolysis) that are in opposite directions to each other and occur simultaneously.

In the industrial scale production of new natural white slow sugar, the direction of esterification is favoured.

At consumption and at the intestinal level it is the opposite, since it is the hydrolysis reaction which is favoured to slowly release (LP) the sugar from the new sugar, namely the sugar ester, and this in accordance with the slow and limited secretion of insulin (FIG. 4).

The sustained release LP of sugar from the novel natural white slow sugar of the present invention at the intestinal level is in agreement and similarity with the slow secretion of insulin in the case of type 2 diabetes (FIG. 4).

The chemical basis of the new natural white slow sugar of this invention is obtained by the chemical mechanism of the synthesis of the new slow sugar:

### Oxidation (FIG. 5-1):

For sugars, there is firstly a reaction of monosaccharides consisting of an oxidation of saccharic acids making it more easy for the attack of primary OH groups to occur and which are generally oxidized entirely to the carboxylic acid state.

The resulting product is a polyhydroxy carboxylic acid called saccharic acid.

For example, nitric acid is a good oxidizing agent for the preparation of saccharic acids.

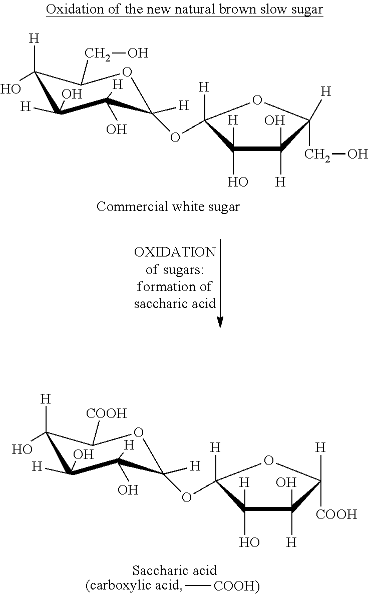

### Esterification (FIG. 5-3):

Hydroxy groups in sugars can be esterified using normal methods (Carboxylic Acid+Alcohol).

Carboxylic acids cause reactions involving the carbon of the carboxyl group, by acid-catalyzed nucleophilic addition.

The best known reaction is Esterification, which is a balanced reaction.

Starting from an equimolar mixture of carboxylic acid and alcohol, we obtain in number of moles 65% of ester and water and 35% of acid and alcohol. This yield can be improved by increasing the concentration of a reactant or by decreasing that of a product (Le Chatelier's Law).

Acid-catalyzed esterification is an important method for the preparation of esters (new slow sugar).

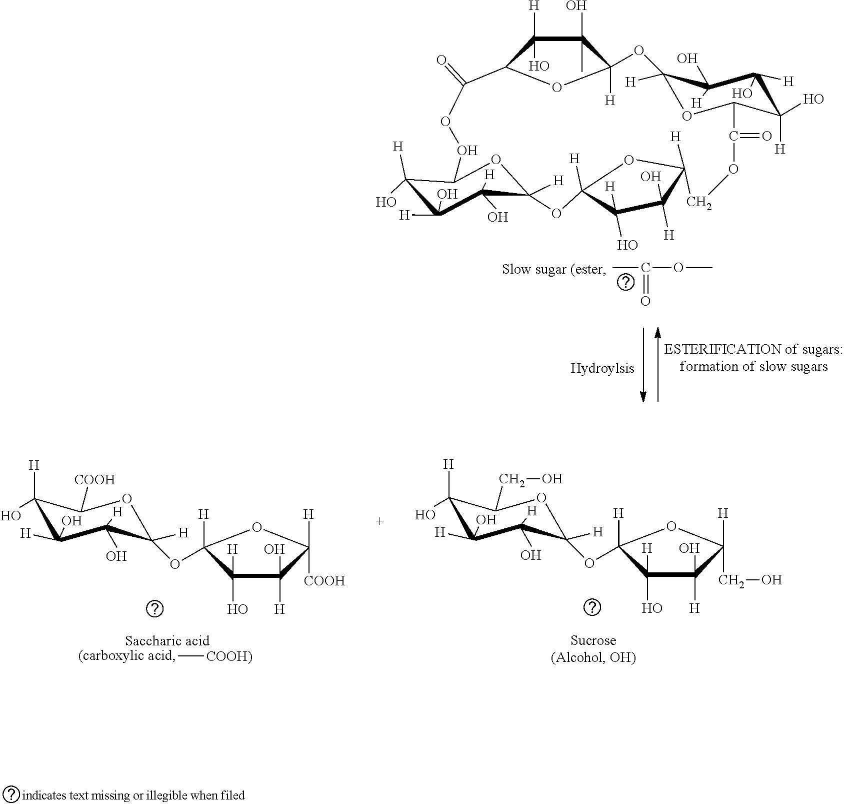

The new natural white slow sugar of the present invention is a slow sugar, because it is obtained and consumed according to the physicochemical characteristics of the “Esterification/hydrolysis” chemical balance that are:
Slow because the release is a prolonged LP
Limited by the reverse reaction
Athermal because the temperature does not modify the equilibrium.
the new natural white slow sugar of the present invention is chemically in the form of esters.

Compared to natural products such as stevia, tannin and apple plants which contain esters, it is concluded that chemicals in the form of esters are not harmful.

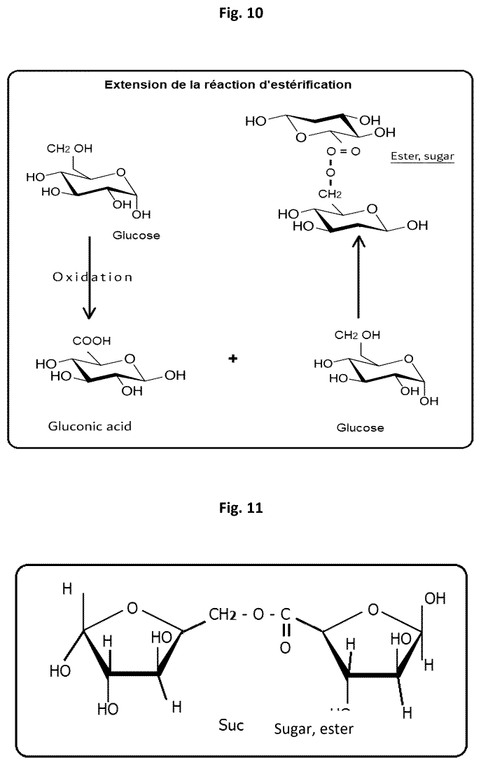

### Extension of the Esterification Reaction (FIG. 10):

Obtaining an ester sugar following the esterification reaction between two types of sugar:
sugar alcohol (function —OH): commercial white sugar.
acidic sugar (function —COOH): saccharic acid.

Following the hydrolysis of sucrose into glucose and fructose, these last two sugars in turn undergo the esterification reaction.

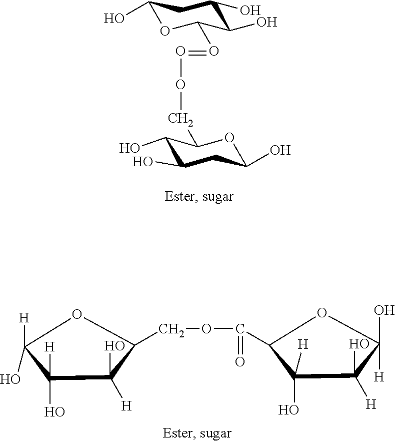

The oxidation of glucose into gluconic acid then the esterification between glucose and gluconic acid into an ester of two glucose molecules:

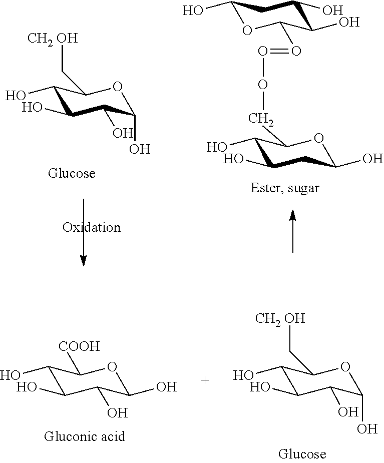

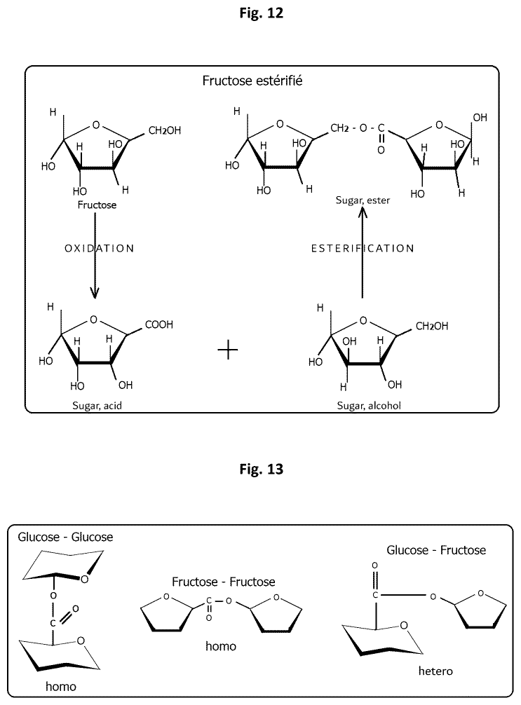

In the same way, the ester sugar for fructose is obtained (FIG. 11).

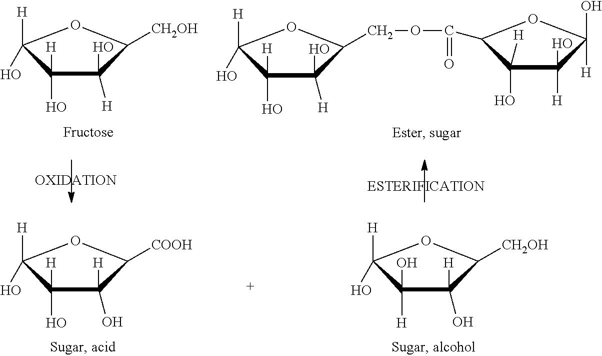

Slow sugars are sugars with a glycemic index of less than 59.

It was long wrongly believed that slow sugars were complex carbohydrates. The new natural white slow sugar obtained from this invention can be used in all food preparations, usually using commercial white natural sugar, such as cakes, pastries, confectionery, sorbets and ice creams, juices, drinks of all kinds, chocolate-based preparations, cocoa-based preparations, caramel-based preparations and others.

The new natural white slow sugar is essential for intense sports that require endurance, whatever their nature, because it is slow to be ingested into the body, ensuring the maintenance of the energy necessary for athletes and therefore it is an anti-cramping product.

In fact, the new natural white slow sugar of this invention is assimilated slowly and therefore remains available for the duration of the effort.

The new natural white slow sugar of this invention does not cause reactive hypoglycemia, on the other hand, commercial natural white sugar has a rapid release and causes a rapid rise in blood sugar levels, which is considered dangerous (hyperglycemia).

Slow sugars are good for your health, these slow sugars are sugars that have a low glycemic index which means that they continuously release energy into the body.

The glycemic index is a criterion for ranking foods containing carbohydrates, based on their effects on blood sugar (glucose levels in the blood) during the two hours following their ingestion. It allows you to compare the glycemic power of each food measured directly during digestion.

A low glycemic index for a food means that the blood glucose level rises only very slightly after the food is digested, as shown in the illustration below.
Low GI≤35: such as dried vegetables, dairy products, meats
Average GI between 35 and 50: such as bananas, apricots.
High GI over 50: such as dates, sweets

So, the intensity with which foods are ingested in increasing blood sugar is expressed by a value called GI.

The new natural white slow sugar of this invention is therefore a slow sugar characterized by its low GI which is measured in the medical analysis laboratory following the protocol of hyperglycemia induced per HGPVO.

Since the new sugar is prepared by chemical esterification, the hydrolysis of esters upon ingestion is a slow reaction that is strongly catalyzed by acids.

In this invention, in the primary and secondary alcohol esters, the mechanism is the reverse of that of acid-catalyzed esterification.

Esterification/hydrolysis are therefore in reaction equilibrium.

The production of the new natural white slow sugar is in accordance with the process described below.

To obtain, for example, a quantity of 10 kg of new natural white granulated or powdered slow sugar, it is necessary to make a mixture of demineralized water with an acid catalyst oxidant in a tank and to heat it to a temperature of 90 to 100 degrees Celsius while stirring.

Gradually add to this mixture existing in the tank which continues to remain on a fire, 10 kg of traditional white commercial sugar for about 10 minutes, mixing simultaneously, with the progressive flow of sugar, using the propeller or blade stirrer, fixed and operating electrically, at a speed of 25 to 35 revolutions/minute, until obtaining a light brown molasses. This operation, called cooking, lasts between 30 and 45 minutes.

After obtaining this molasses, the fire is then extinguished and this molasses undergoes drying with stirring at a speed of 30 to 50 revolutions/min, under the effect of blowing fresh air followed by blowing hot air for a period of 30 to 35 min.

At the end of this drying operation, this molasses then becomes a new natural white granulated slow sugar which will be subjected to sieving to obtain the new sugar with a uniform granulometry. Sieving results in a weight loss of 300 to 400 grams on the initial 10 kg, identifiable by the final weighing of the new natural white slow sugar obtained from the present invention.

If there is a need to produce new natural white slow sugar in powder form, the manufacturing process described must be completed by grinding this new granulated natural white slow sugar obtained after sieving.

The preparation of larger quantities follows the same procedure in terms of time, temperatures and speed listed above, at all stages regardless of the weight of the new natural white slow sugar to be produced, provided that one or more tanks are available to measure the desired quantity to be produced, equipped with an adequate number of agitators capable of reaching, by agitation, the entire quantity existing in the tank(s). It is also possible to produce a large quantity by using several tanks with a capacity of 10 or 20 kg or more, operated by a control triggering simultaneous production.

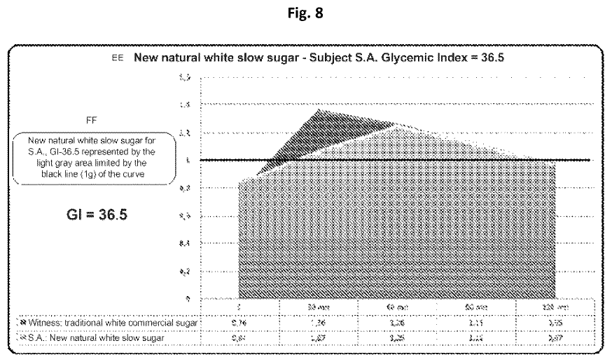

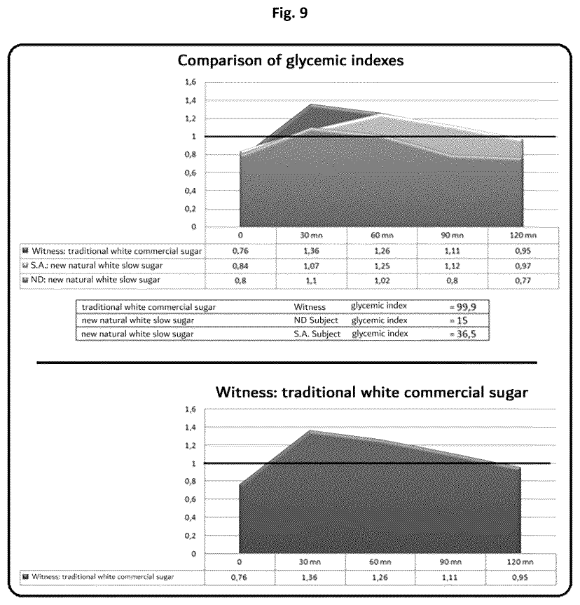

The new natural white slow sugar of this invention has a glycemic index between GI 15 and 36.5 represented by the full area at the level of the curves (FIG. 7) and (FIG. 8). The new natural white granulated or powdered slow sugar of this invention is dietetic, calorie-free, anti-obesity and prevents dental caries.

It is also antidiabetic and therefore prevents retinopathy, and nephropathy, among others, because the new natural white slow sugar of the present invention is esterified and it is characterized by a prolonged release LP under the effect of hydrolysis, the reverse chemical reaction of esterification.

Its consumption is therefore intended for healthy subjects and also for type 2 diabetics. Thus a blood HbA1C level of 7% corresponds to an average blood sugar level of 1.5 g per litre of blood and 1% more or less HbA1C represents an average increase or decrease in blood sugar level of 0.30 g/litre.

For the new natural white granulated or powdered slow sugar of this invention the following result is obtained:

$$\frac{0.3 \times 0.3}{1} = 0.072 ,$$

which clearly shows that the new natural white slow sugar decreases HbA1c level by 7.2%

For traditional white commercial sugar and for comparison purposes we obtain the following result:

$$\frac{0.32 \times 0.3}{1} = 0.092 ,$$

which indicated that commercial white natural sugar increases HbA1c level by 9.2%

Slow sugars have long been considered to be replacement sugars and not substitute sugars or fake sugars and are often presented in liquid form, except that their preparation does not conform to a manufacturing process resembling the process of this invention, which is based on the chemical equilibrium “esterification/hydrolysis”.

The physicochemical characteristics of the new natural white granulated or powdered slow sugar of this invention, obtained from the following analyses carried out over 12 months, are shown in the following table of values, which confirms the chemical stabilization of the new sugar. This new sugar is a concentrated medium that can be stored for a long time without deteriorating.

| Parameters | | | Dry | Total | | |
|---|---|---|---|---|---|---|
| Results | pH | Carbohydrates | extract | Ash | Lead | Cadmium |
| January 2021 | 4.86 | 92.5 | 99.3 | 0.1 | <0.01 | 0.1 |
| February 2021 | 4.90 | 92.3 | 99.3 | 0.1 | <0.01 | 0.1 |
| March 2021 | 4.40 | 93.8 | 99.3 | 0.1 | <0.01 | 0.1 |
| April 2021 | 4.93 | 92.2 | 99.4 | 0.3 | <0.01 | 0.3 |
| May 2021 | 4.92 | 93.9 | 99.3 | 0.1 | <0.01 | 0.1 |
| June 2021 | 4.90 | 92.2 | 99.3 | 0.1 | <0.01 | 0.3 |
| July 2021 | 4.91 | 92.4 | 99.2 | 0.1 | <0.01 | 0.1 |
| August 2021 | 4.88 | 92.0 | 99.3 | 0.1 | <0.01 | 0.1 |
| September 2021 | 4.93 | 92.2 | 99.3 | 0.1 | <0.01 | 0.1 |
| October 2021 | 4.91 | 93.8 | 99.3 | 0.1 | <0.01 | 0.1 |
| November 2021 | 4.96 | 92.2 | 99.3 | 0.1 | <0.01 | 0.1 |
| December 2021 | 4.89 | 92.3 | 99.3 | 0.1 | <0.01 | 0.3 |

The new natural white granulated or powdered slow sugar is nothing other than an equimolar mixture of glucose and fructose obtained by enzymatic or chemical hydrolysis of sucrose.

Oxidative hydrolysis of sucrose, which normally occurs partially, has produced completely new sucrose.

The new natural white granulated or powdered slow sugar of the present invention is characterized by a refractive index of 1.3412 and a polarization index according to the Z° unit of 64.9.

The glycemic index was measured following the protocol of Oral Hyperglycemia (OGTT), by making a comparison based on analyses of the new natural white slow sugar repeated on two subjects, and such analyses made it possible to note low glycemic indices compared to traditional commercial white sugar taken as a control and whose glycemic index is very high (FIG. 9).

The molecules of the esterified sugars of the new natural white slow sugar, that is to say esterified Sucrose, esterified Glucose and esterified Fructose in double molecule, homo or hetero are really and inevitably chemically synthesizable, namely: Glucose-Glucose, Fructose-Fructose, Glucose-Fructose and Sugar-Sugar.

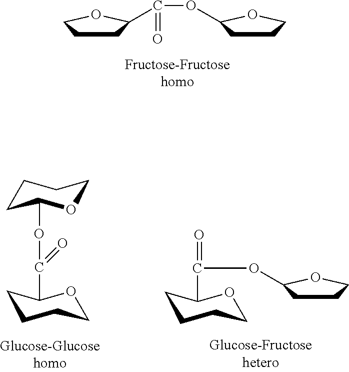

This is argued and proven by the results obtained from the Esterification Indexes IE, calculated by Saponification Index IS and Acid Index IA (IE=IS−IA) according to the French Pharmacopoeia tenth edition.

| ACID | | SAPONIFICATION | | ESTER |
|---|---|---|---|---|
| INDEX | | INDEX | | INDEX |
| n(mL) | 1.35 | n1(mL) | 1.2 | IE = IS − IA |
| m(g) | 10 | n2(mL) | 1.31 | IS = 3.0855 |
| | | m(g) | 1 | IA = 0.75735 |

| | | |
|---|---|---|
| IA = 0.75735 | IS = 3.0855 | IS = 2.32815 |

All the esterified sugar molecules designated in this invention bear the name SATTAR and are obtained by:
OXIDATION: All primary OH groups of sucrose, glucose and fructose are attacked more easily and are usually oxidized completely to the carboxylic acid state.
ESTERIFICATION: to subsequently ensure esterification, these molecules react with the sugar alcohol molecules, in homo-molecular or/and corresponding hetero-molecular (FIG. 5-2a) and (FIG. 5-2b).

1. A new natural white granulated or powdered slow sugar, which is obtained by a manufacturing process based on esterification starting from commercial white natural sugar through oxidation carried out by an acid oxidant and esterification of the sugars by the same acid oxidant catalyst in order to obtain esters in accordance with the new molecule below;

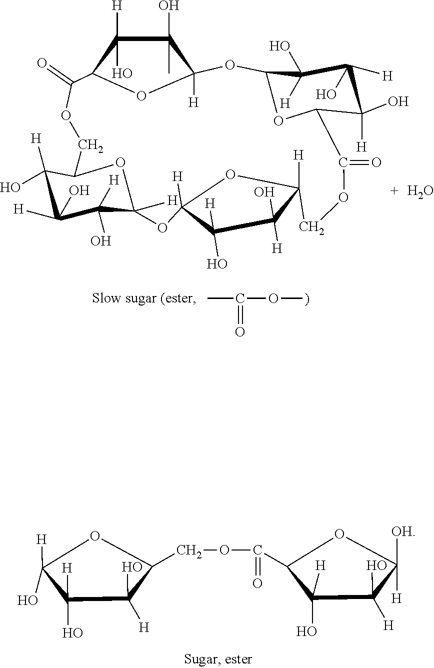

2. The new natural white granulated or powdered slow sugar of claim 1 retaining the sweetening power of commercial white natural sugar and characterized by its low glycemic index between GI 15 and 36.5.

3. The new natural white granulated or powdered slow sugar of claim 1 characterized by a refractive index of 1.3412 and a polarization unit Z° 64.9.

4. The new natural white slow sugar, of claim 1 which can be consumed by type 2 diabetic subjects.

5. The new natural white slow sugar of claim 1 is anti-obesity by the effect of non-storage and the elimination of the surplus of sugar consumed and this, thanks to the chemical balance esterification/hydrolysis acting in accordance with the slow secretion of insulin in the body.

6. The new natural white slow sugar of claim 1 in that it is dietetic and without caloric intake, preventing dental caries by blocking the alcohol function (—OH) and can be consumed by diabetic subjects to slow down or/and prevent retinopathy and nephropathy and other pathological complications.

7. The new natural white slow sugar of claim 1 in liquid solution, syrup, paste, cream, honey or caramel.

8. The process for manufacturing of new natural white granulated or powdered slow sugar of claim 1 comprising the following:
Mix two liters of demineralized water with an acid oxidant
Heat and stir for 10 minutes in a bowl over a stove to reach 90 to 100 degrees Celsius
Add 10 kg of commercial white natural sugar to the mixture
Cook while stirring at a speed of 25 to 35 rpm
Dry the molasses obtained in fresh air, then in hot air for 30 to 45 minutes, stirring at a speed of 30 to 50 rpm.

9. A new natural white slow sugar recommended for athletes practicing intense sports, for its slow character ensuring a prolonged release and because it carries characteristics offering endurance and preservation of muscles against cramps.

10. This is a property of the new natural white granulated or powdered slow sugar used as raw material for the manufacture of products in liquid, pasty, creamy, honeyed or caramelized forms or other forms of claim 1 for the preparation or production of pharmaceutical and parapharmaceutical products as well as for body care, well-being and cosmetic products.

11. The new natural white slow sugar of claim 1 obtained by the chemical balance esterification/hydrolysis having the slow, limited and athermal characteristics.

12. The new natural white slow sugar of claim 1 obtained by a reaction between sugar alcohol (sucrose) and an acidic sugar (saccharic acid) which is obtained by oxidation of the sugar alcohol.

13. The new natural white slow sugar of claim 1 prepared using the same acid oxidizing catalyst jointly for the oxidation of the sugar and for the esterification.

14. A new natural white slow sugar, slow when ingested into the intestine of the body characterized by its prolonged release LP in accordance with the secretion of insulin.

15. A new natural white slow sugar chemically in the form of esters, with regard to glucose, in accordance with the following molecule:

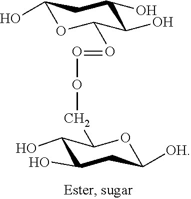

16. A new natural white slow sugar chemically in the form of esters, regarding fructose, in accordance with the following molecule:

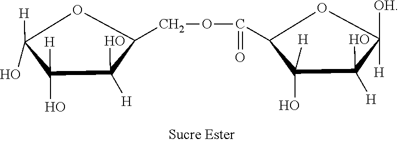

17. The esterification of two sugars of each molecule separately or hetero i.e. esterification of the glucose molecule and the fructose molecule together

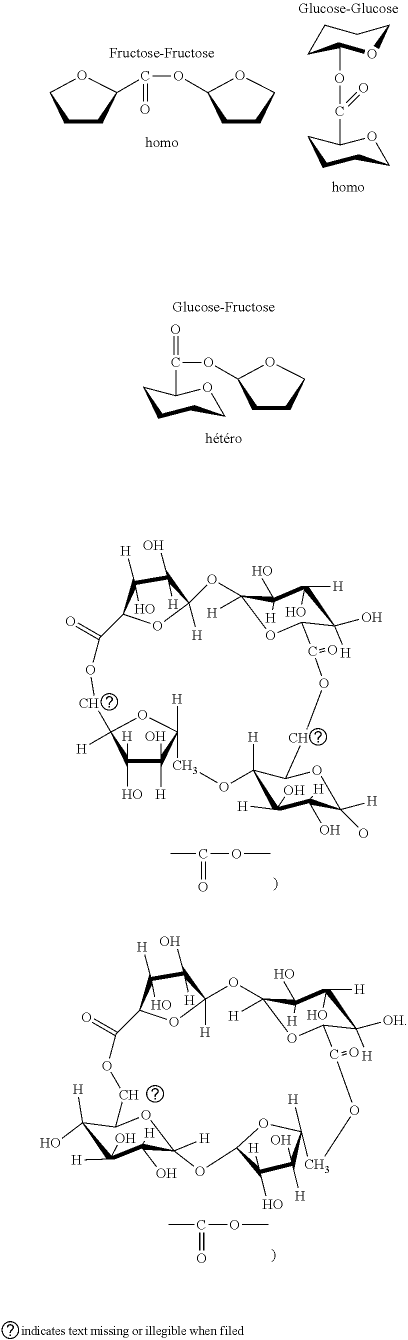

18. The oxidation of glucose into gluconic acid, then esterification between glucose and gluconic acid into an ester of two glucose molecules: 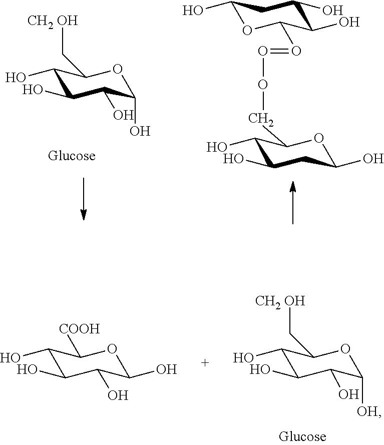
and
in the same way, the ester sugar for fructose is obtained

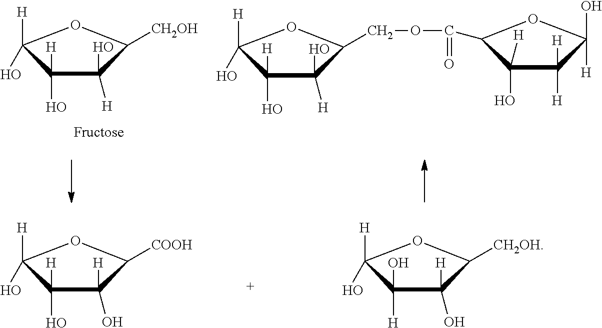

19. The new natural white granulated or powdered slow sugar of claim 2 characterized by a refractive index of 1.3412 and a polarization unit Z° 64.9.

20. The new natural white slow sugar, of claim 2 which can be consumed by type 2 diabetic subjects.
